(19) United States
(12) Patent Application Publication
(10) Pub. No.: US 2017/0020995 A1
(43) Pub. Date: Jan. 26, 2017

(54) Serine Protease Primary Hemostatic Agent

(71) Applicant: Leon Wortham, Chattanooga, TN (US)

(72) Inventor: Leon Wortham, Chattanooga, TN (US)

(21) Appl. No.: 14/808,675

(22) Filed: Jul. 24, 2015

(51) Int. Cl. A61K 38/48

(52) U.S. Cl. CPC A61K 38/4833; A61K 38/4846; C12Y 304/21005; C12Y 304/21021; C12Y 304/21022; C12Y 304/21006

(57) ABSTRACT

Techniques and compositions are shown for stabilizing labile serine proteases in order to maintain their enzymatic properties over a long extended period and the utilization of stable serine proteases as a one component primary hemostatic agent.

### FIELD OF THE INVENTION

The invention relates to serine protease hemostatic agents. Disclosed are techniques for stabilising labile serine proteases in order to maintain their enzymatic properties over a long extended period and the utilisation of stable serine proteases as a one component primary hemostatic agent.

### BACKGROUND OF THE INVENTION

Bleeding is a major cause of morbidity and mortality in wounds. After a traumatic injury, hemorrhage is responsible for over 35% of pre-hospital deaths and over 40% of deaths within the first 24 hours, second only to the rates of death due to severe central nervous system injury. A cascade of life-threatening medical problems can begin with severe hemorrhage, and many of these occur simultaneously: 1) hemorrhage, 2) impaired resuscitation, 3) shock, 4) inflammation and 5) coagulopathy. The severity of each problem is commonly associated with the extent of overall blood loss. Low blood pressure due to blood loss indicates immediate complications, including the incidence of multiple organ failure and life-threatening infections.

Hemostatic agents have been used in various surgical disciplines as an adjunct to control bleeding in surgical wounds. There are several types of hemostatic agents: collagen, gelatin, starch, kaolin, hydro-absorbent agents, cyanoacrylic, thrombin, and fibrin glues. Presently, the most effective adjunct hemostatic agents are the fibrin glues (sealants). The fibrin glue is a two component system, consisting of fibrinogen, or collagen, or a hydro gel as one component, and thrombin in the second component as an activator. The mixture is only effectively delivered by a syringe or as a spray when connected to a pressure pump, and produces a highly viscous material that obscures the bleeding site. The mechanism of the fibrin glue places a physical barrier over the open blood vessel, impeding blood from escaping, and allowing the patient's own coagulation system to form a fibrin clot. This method is limited in its effectiveness. An application of such fibrin glue is limited to venous bleeds that have low vascular pressure, and in patients with normal clotting times it will stop the bleeding in 1-3 minutes. In patients with arterial hemorrhages or abnormal clotting times, due to anticoagulant medication or factor deficiencies, the fibrin glue is ineffective.

The primary methods to control bleeding are cauterization, sutures, staples, tourniquet, and direct pressure. Although effective to control a wide range of hemorrhages, they also face challenges that limit their ability to arrest the bleed, or their application is more deleterious than beneficial. For example, injured blood vessels that retract below the surface or behind other tissues are inaccessible to standard surgical modalities to arrest the bleed. Another example, cauterization seals the blood vessel by burning the tissue, which is irreversible. For instance, patients with cardio-vascular deficiency or diabetes are predisposed to develop ulcers in their extremities, due to blood insufficiency. The loss of vascular tissue from cauterization only further compounds these patients' overall health. Still another challenge in using these primary hemostatic tools, is their efficacy on coagulopathy patients. Effective use of these primary hemostatic controls may require the anticoagulant medication to be withdrawn before surgery, or the infusion of fresh frozen plasma to the factor deficient patient. Either of these steps place the patient at a higher risk for an adverse event.

An alternative method is the development of a topical stable one component liquid hemostatic agent, composed of serine proteases: Factors II, VII, IX, X, and agar, a linear complex sugar made from beta-galactopyranose linked to 3,6-anhydro-L-galactopyranose shown in FIG. 1. These serine protease enzymes are labile and are quickly hydrolyzed at room temperature. As a result, other prothrombin complex concentrates (containing Factors II, VII, IX, and X) are lyophilized, and must be reconstituted with liquid. The stability of coagulation factors within hemostatic formulations is important, as they are expensive and the longer the shelf fife, the less formulation that must be discarded due to expiration. Furthermore, there are inherent problems associated with a lyophilized product that are either reduced or eliminated in a liquid product. These include quantitation errors, the introduction of impure water or microbial contamination during reconstitution.

The three equatorial hydrogen atoms on the 3,6-anhydro-L-galactose residues constrain the agarose molecules to form a helix. The interaction of the helixes coordinates water molecules, causing the formation of a gel, and preventing the hydrolization of the serine proteases. When there are a group of serine proteases together, the molecules will act upon each other with resulting degradation and loss of coagulation activity. In these reactions, one serine protease is the “enzyme” and another is the “substrate”.

In the process of hydrolysis one serine protease, the “enzyme” forms a stable complex bound to another serine protease, the “substrate.” When free water molecules are present, a water molecule will replace the “substrate” from the “enzyme” resulting in cleavage of the “substrate” and reformation of the “enzyme”. However, when agar is coordinating all the water molecules, the enzyme and substrate will remain in the stable conformation and the time to cleave each other is greatly extended.

The resulting stable liquid has primary hemostatic properties. It is capable of arresting all forms of hemorrhages within seconds, even in patients on anticoagulant therapy or in hemophiliac patients, without the loss of tissue. Accelerated hemostasis occurs through two separate processes working in tandem to seal the bleeding wound in seconds. In one process, agar will cross-link with the ions of platelet phospholipids at the glycoprotein IIb/IIIa site and cations from amine groups in fibrinogen/fibrin monomers and tissue proteins, forming an α-1,6-linked galactophospho and α-1,6-galactoamine bond, bridging both the platelets and fibrinmonomers, sealing the wound faster.

In the second process, the aggregation of platelets causes a change in the membrane surface, activating the platelets to release the contents of stored granules into the blood plasma, initiating the coagulation cascade system to produce a fibrin clot. The serine proteases in the primary hemostatic agent catalyze both the tissue factor (extrinsic) and the contact activation (intrinsic) pathways that make up the normal coagulation cascade system. The activated serine proteases from the hemostatic agent accelerate the specific sites within the coagulation system, to generate a thrombin burst that transforms fibrinogen to fibrin, the building block of a fibrin clot that seals the injured blood vessel.

As illustrated in FIG. 2, the hemostatic mechanism consists of a series of activation stages in which circulating coagulation factors are converted in sequence from inactive precursors to activated forms. Activation of the hemostatic mechanism is specific for each of the two activation pathways in clot formation: the extrinsic and the intrinsic systems. In the extrinsic system, factor VII is activated by tissue thromboplastin and forms a complex by binding to factor VII and phospholipid. Calcium ions are introduced and the inactive factor X is transformed into the active factor Xa. The intrinsic pathway is activated by the binding of factor XII to subendothelial collagen. Activated factor XII forms a complex with factor XI, thus activating it to XIa. Factor XIa then activates factor IX, which forms a new complex with factor VIII, phospholipids and calcium ions. The latter complex activates factor X to Xa.

After the activation of factor X both the extrinsic and intrinsic systems merge together and follow a common pathway to clot formation. Factor Xa forms a complex (prothrombin-converting complex) with factor V, phospholipids and calcium ions, which then activates prothrombin to thrombin. The latter is a proteolytic enzyme, not normally present in plasma, that converts fibrinogen into soluble fibrin monomer. In the course of this conversion, fibrinopeptides A and B are released. Fibrin monomers polymerize spontaneously in the presence of calcium to form a soluble fibrin clot.

The serine protease agents described herein are capable of utilising both the intrinsic and extrinsic pathways to support the promotion of clot formation in only seconds, even in the presence of coagulopathies.

The serine protease primary hemostatic agent is more effective than cauterization to arrest the hemorrhage faster and reduce the amount of blood loss, as seen in three animal models: rabbit, dog, and the pig, with a mean 44% faster clotting time and a 65% reduction of blood loss for severe to life-threatening forms of hemorrhage (Tables 1-4).

TABLE 1
Hemostasis in the Rabbit and Dog

| | Rabbit | | | | Dog | | | |
|---|---|---|---|---|---|---|---|---|
| | | Serine | | Cauterization | | Serine | | Cauterization |
| Organ/Tissue | n | Protease(sec) | n | Control(sec) | n | Protease(sec) | n | Control(sec) |
| Liver | 48 | 3.21(∀0.16) | 2 | 18.43(∀1.87) | 8 | 2.87(∀0.14) | 2 | 24.0(∀2.83) |
| Spleen | 14 | 2.80(∀0.07) | 2 | 5.25(∀0.17) | 8 | 2.44(∀0.10) | 2 | 7.95(∀0.49) |
| Kidney | 8 | 2.92(∀0.04) | 2 | 6.93(∀1.08) | 2 | 2.83(∀0.05) | 2 | 7.75(∀0.35) |
| Mandible/Maxilla | — | — | — | — | 2 | 2.85(∀0.21) | 2 | 2.30(∀0.14) |
| Arteries: | | | | | | | | |
| Hepatic | 12 | 4.95(∀0.11) | 2 | 4.60(∀0.14) | 3 | 5.67(∀0.60) | 2 | 4.83(∀0.35) |
| Spleenic | 6 | 4.33(∀0.06) | 2 | 4.90(∀0.14) | 2 | 4.60(∀0.56) | 2 | 4.35(∀0.21) |
| Testicular | 2 | 3.26(∀0.08) | 2 | 4.35(∀0.21) | 2 | 3.60(∀0.56) | 2 | 4.30(∀0.28) |
| Ovarian | — | — | — | — | 2 | 3.85(∀0.21) | 2 | 4.55(∀0.07) |
| Root Canal | — | — | — | — | 2 | 1.40(∀0.14) | 2 | 153(V22.6) |

In the rabbit, the overall mean time to hemostasis for the 7 surgical procedures performed was 3.43±0.69 seconds with serine protease (n=90), compared to 7.41±5.22 seconds (n=12) in the cauterization control group, a significant faster clotting time (P=<0.0001).

Serine protease also demonstrated to outperform cauterization in 9 surgical procedures in the dog, by yielding an overall mean time to hemostasis of 3.15±1.09 seconds (n=31) to 23.67±47.47 seconds (n=18) in the cauterization group (P=0.0192).

Assessment of the efficacy study in the rabbit model showed a 57.2% shorter time to hemostasis over cauterization/ligation, and a significantly 117% faster clotting time over the standard primary method to control bleeding in the dog model.

TABLE 2
Blood Loss in the Rabbit and Dog

| | Rabbit | | | | Dog | | | |
|---|---|---|---|---|---|---|---|---|
| | | Serine | | Cauterization | | Serine | | Cauterization |
| Organ/Tissue | n | Protease(ml) | n | Control(ml) | n | Protease(ml) | n | Control(ml) |
| Liver | 48 | 2.67(∀0.34) | 2 | 4.85(∀0.35) | 8 | 3.10(∀0.85) | 2 | 5.00(∀0.42) |
| Spleen | 14 | 1.85(∀0.73) | 2 | 2.75(∀0.07) | 8 | 1.78(∀0.56) | 2 | 3.85(∀0.49) |
| Kidney | 8 | 3.22(∀0.68) | 2 | 4.90(∀0.28) | 2 | 2.20(∀0.28) | 2 | 2.25(∀0.49) |
| Mandible/Maxilla | — | — | — | — | 2 | 1.20(∀0.42) | 2 | 1.05(∀0.35) |
| Arteries: | | | | | | | | |
| Hepatic | 12 | 6.43(∀1.85) | 2 | 5.86(∀1.40) | 3 | 5.25(∀1.92) | 2 | 7.38(∀0.94) |
| Spleenic | 6 | 5.50(∀1.12) | 2 | 5.73(∀1.55) | 2 | 4.62(∀1.12) | 2 | 6.44(∀0.52) |
| Testicular | 2 | 3.80(∀0.56) | 2 | 3.60(∀0.42) | 2 | 2.20(∀0.28) | 2 | 2.10(∀0.28) |
| Ovarian | — | — | — | — | 2 | 2.35(∀0.64) | 2 | 2.75(∀0.21) |
| Root Canal | — | — | — | — | 2 | 0.45(∀0.07) | 2 | 8.80(∀0.85) |

A significant reduction of blood loss in both the rabbit and dog models was observed with serine protease over cauterization, with a mean 3.31±1.48 ml and 2.60±1.30 ml compared to 4.62±1.16 ml and 4.40±2.59 ml, respectively.

Assessment of the safety of serine protease demonstrated a 16.7% reduction of blood loss in the rabbit, and a two-fold (37%) reduction of blood loss over the cauterization group in the dog.

TABLE 3
Hemostasis in the Pig

| Surgical Procedure | n | Serine Protease (sec) | n | Control (sec) |
|---|---|---|---|---|
| Aorta resection | 3 | 69.33 ∀ 10.07 | 3 | 86.33 ∀ 10.07 |
| Partial nephrectomy | 5 | 24.20 ∀ 12.85 | 5 | 40.60 ∀ 7.02 |
| Bowel resection | 2 | 27.50 ∀ 5.24 | 2 | 36.00 ∀ 5.66 |
| Cholecystectomy | 7 | 28.71 ∀ 6.78 | 7 | 44.60 ∀ 6.87 |
| Urethra resection | 4 | 20.00 ∀ 4.40 | 4 | 36.75 ∀ 8.22 |

Table 3: In 5 surgical procedures in the pig, serine protease achieved a mean 33.95±7.87 seconds clotting time, compared to a mean 48.86±7.57 seconds for the cauterization surgical modality to control bleeding, with a 44% improved clotting time.

TABLE 4
Blood Loss in the Pig

| Surgical Procedure | n | Serine Protease (ml) | n | Control (ml) |
|---|---|---|---|---|
| Aorta resection | 3 | 128.67 ∀ 13.20 | 3 | 226.67 ∀ 9.07 |
| Partial nephrectomy | 5 | 61.20 ∀ 8.76 | 5 | 83.40 ∀ 4.67 |
| Bowel resection | 2 | 19.00 ∀ 5.66 | 2 | 29.50 ∀ 3.54 |
| Cholecystectomy | 7 | 56.43 ∀ 7.98 | 7 | 98.00 ∀ 7.00 |

Table 4: Blood loss was significantly less in the serine protease group, with a mean 69.62±8.90 ml versus 109.39±6.07 ml in the cauterization/ligation group, a 57% reduction in blood loss

The significant reduction in hemostasis and blood loss is due to the application of the two devices. For example, a 5 cm incision is made, resulting in five individual bleeds within the wound. To arrest those bleeds with an electric cautery, each bleed is cauterized one at a time, for a collective time to hemostasis of 5 seconds. On the other hand, the serine protease hemostatic agent, being a liquid, floods the wound and captures all of the individual bleeds at one time, yielding a 1-2 second hemostasis time.

The composition of the serine protease hemostatic agent also provides its ability to off-set the effects of platelet inhibitors: Plavix, Aggrenox, aspirin, that bind to sites other than where agar attaches to the phospholipids at the glycoprotein IIb/IIIa platelet site, and the anticoagulant medications: warfarin and heparin, that bind Factor VII and Factor IX, respectively, by providing untreated Factors VII and IX.

The serine protease hemostatic agent's mechanism to arrest the hemorrhage does not create an inflammatory process, allowing the growth factors Interluken-10 and tissue growth factor β-1 to appear at the wound site within 24 hours to promote wound healing, compared to 5 and 7 days, respectively, for the fibrin glue and cauterization treatments.

Serine proteases are very labile, with a shelf-life of 2-24 hours. A unique stabiliser is added to the primary hemostatic agent that prevents oxidation, hydolyization, degradation to the enzymes, and allows the device to be subject to repetitive freeze-thaw cycles without losing its activity (Table 5). In this study the level of coagulation factors present is measured in International Units, IU, where 1 IU/ml is representative of normal human plasma. The measurements of coagulation factors where made using Factor assay methodology. The general principles of all functional clotting factor assays are the same and involve plotting clotting time (from either a PT or an APTT depending upon which factor is being assayed) against sample dilution. The degree of correction of the clotting time when the plasma is added to a clotting system specifically deficient in the clotting factor to be measured allows the level of that clotting factor to be determined e.g. A factor VIII deficient plasma is used to assay the level of FVIII. In each case a reference plasma with a known level of a specific clotting factor is required.

### TABLES 5A and 5B

### Stability Study

TABLE 5A
Accelerated

| 37° C. | Lot 1 | Lot 2 | Lot 3 | 25° C. | Lot 1 | Lot 2 | Lot 3 |
|---|---|---|---|---|---|---|---|
| Day | Activity | Activity | Activity | Day | Activity | Activity | Activity |
| 1 | 3030 IU/ml | 3100 IU/ml | 3012 IU/ml | 1 | 3070 IU/ml | 3094 IU/ml | 3048 IU/ml |
| 2 | 2988 IU/ml | 3010 IU/ml | 2970 IU/ml | 2 | 3064 IU/ml | 3090 IU/ml | 3044 IU/ml |
| 3 | 2940 IU/ml | 2974 IU/ml | 2926 IU/ml | 3 | 3056 IU/ml | 3082 IU/ml | 3040 IU/ml |
| 4 | 2900 IU/ml | 2932 IU/ml | 2884 IU/ml | 4 | 3050 IU/ml | 3076 IU/ml | 3032 IU/ml |
| 5 | 2860 IU/ml | 2890 IU/ml | 2840 IU/ml | 5 | 3042 IU/ml | 3072 IU/ml | 3024 IU/ml |
| 6 | 2686 IU/ml | 2716 IU/ml | 2662 IU/ml | 6 | 3034 IU/ml | 3066 IU/ml | 3014 IU/ml |
| 7 | 2418 IU/ml | 2438 IU/ml | 2398 IU/ml | 7 | 3028 IU/ml | 3058 IU/ml | 3002 IU/ml |
| 8 | 2172 IU/ml | 2194 IU/ml | 2150 IU/ml | 8 | 3022 IU/ml | 3050 IU/ml | 2994 IU/ml |
| 9 | 1952 IU/ml | 1978 IU/ml | 1936 IU/ml | 9 | 3018 IU/ml | 3044 IU/ml | 2988 IU/ml |
| 10 | 1750 I/ml | 1772 IU/ml | 1738 IU/ml | 10 | 3010 IU/ml | 3038 IU/ml | 2984 IU/ml |
| | | | | 11 | 3002 IU/ml | 3028 IU/ml | 2976 IU/ml |
| | | | | 12 | 2994 IU/ml | 3020 IU/ml | 2970 IU/ml |
| | | | | 13 | 2980 IU/ml | 3004 IU/ml | 2968 IU/ml |
| | | | | 14 | 2972 IU/ml | 2992 IU/ml | 2960 IU/ml |
| | | | | 15 | 2962 IU/ml | 2986 IU/ml | 2952 IU/ml |
| | | | | 16 | 2950 IU/ml | 2980 IU/ml | 2944 IU/ml |
| | | | | 17 | 2946 IU/ml | 2978 IU/ml | 2938 IU/ml |
| | | | | 18 | 2940 IU/ml | 2970 IU/ml | 2934 IU/ml |
| | | | | 19 | 2934 IU/ml | 2966 IU/ml | 2930 IU/ml |
| | | | | 20 | 2930 IU/ml | 2958 IU/ml | 2924 IU/ml |
| | | | | 21 | 2922 IU/ml | 2950 IU/ml | 2920 IU/ml |
| | | | | 22 | 2914 IU/ml | 2942 IU/ml | 2912 IU/ml |
| | | | | 23 | 2906 IU/ml | 2938 IU/ml | 2904 IU/ml |
| | | | | 24 | 2900 IU/ml | 2930 IU/ml | 2898 IU/ml |
| | | | | 25 | 2892 IU/ml | 2922 IU/ml | 2892 IU/ml |
| | | | | 26 | 2884 IU/ml | 2914 IU/ml | 2884 IU/ml |
| | | | | 27 | 2878 IU/ml | 2904 IU/ml | 2876 IU/ml |
| | | | | 28 | 2870 IU/ml | 2896 IU/ml | 2870 IU/ml |
| | | | | 29 | 2860 IU/ml | 2890 IU/ml | 2862 IU/ml |
| | | | | 30 | 2852 IU/ml | 2880 IU/ml | 2852 IU/ml |
| | | | | 31 | 2842 IU/ml | 2872 IU/ml | 2840 IU/ml |
| | | | | 32 | 2830 IU/ml | 2862 IU/ml | 2826 IU/ml |
| | | | | 33 | 2814 IU/ml | 2848 IU/ml | 2812 IU/ml |
| | | | | 34 | 2796 IU/ml | 2830 IU/ml | 2774 IU/ml |
| | | | | 35 | 2776 IU/ml | 2810 IU/ml | 2756 IU/ml |
| | | | | 36 | 2752 IU/ml | 2786 IU/ml | 2732 IU/ml |
| | | | | 37 | 2476 IU/ml | 2508 IU/ml | 2460 IU/ml |
| | | | | 38 | 2228 IU/ml | 2256 IU/ml | 2214 IU/ml |
| | | | | 39 | 2002 IU/ml | 2028 IU/ml | 1990 IU/ml |
| | | | | 40 | 1800 IU/ml | 1828 IU/ml | 1790 IU/ml |
| | | | | 41 | 1616 IU/ml | 1640 IU/ml | 1602 IU/ml |
| | | | | 42 | 1452 IU/ml | 1478 IU/ml | 1438 IU/ml |
| | | | | 43 | 1302 IU/ml | 1326 IU/ml | 1992 IU/ml |
| | | | | 44 | 1168 IU/ml | 1192 IU/ml | 1790 IU/ml |
| | | | | 45 | 1044 IU/ml | 1066 IU/ml | 1604 IU/ml |
| 2-4° C. | Lot 1 | Lot 2 | Lot 3 | −10° C. | Lot 1 | Lot 2 | Lot 3 |
| Day | Activity | Activity | Activity | Day | Activity | Activity | Activity |
| 1 | 3076 IU/ml | 3100 IU/ml | 3054 IU/ml | 1 | 3076 IU/ml | 3100 IU/ml | 3060 IU/ml |
| 2 | 3080 IU/ml | 3094 IU/ml | 3052 IU/ml | 2 | 3076 IU/ml | 3100 IU/ml | 3060 IU/ml |
| 3 | 3078 IU/ml | 3098 IU/ml | 3056 IU/ml | 3 | 3076 IU/ml | 3100 IU/ml | 3060 IU/ml |
| 4 | 3078 IU/ml | 3096 IU/ml | 3060 IU/ml | 4 | 3076 IU/ml | 3100 IU/ml | 3060 IU/ml |
| 5 | 3076 IU/ml | 3098 IU/ml | 3060 IU/ml | 5 | 3076 IU/ml | 3100 IU/ml | 3060 IU/ml |
| 6 | 3078 IU/ml | 3100 IU/ml | 3056 IU/ml | 6 | 3076 IU/ml | 3100 IU/ml | 3060 IU/ml |
| 7 | 3078 IU/ml | 3098 IU/ml | 3054 IU/ml | 7 | 3076 IU/ml | 3100 IU/ml | 3060 IU/ml |
| 8 | 3076 IU/ml | 3100 IU/ml | 3054 IU/ml | 8 | 3078 IU/ml | 3100 IU/ml | 3060 IU/ml |
| 9 | 3080 IU/ml | 3100 IU/ml | 3056 IU/ml | 9 | 3076 IU/ml | 3100 IU/ml | 3060 IU/ml |
| 10 | 3076 IU/ml | 3096 IU/ml | 3056 IU/ml | 10 | 3076 IU/ml | 3100 IU/ml | 3060 IU/ml |
| 11 | 3076 IU/ml | 3098 IU/ml | 3058 IU/ml | 11 | 3076 IU/ml | 3100 IU/ml | 3060 IU/ml |
| 12 | 3078 IU/ml | 3098 IU/ml | 3054 IU/ml | 12 | 3076 IU/ml | 3100 IU/ml | 3058 IU/ml |
| 13 | 3080 IU/ml | 3096 IU/ml | 3054 IU/ml | 13 | 3076 IU/ml | 3100 IU/ml | 3060 IU/ml |
| 14 | 3074 IU/ml | 3094 IU/ml | 3052 IU/ml | 14 | 3076 IU/ml | 3100 IU/ml | 3060 IU/ml |
| 15 | 3076 IU/ml | 3096 IU/ml | 3052 IU/ml | 15 | 3076 IU/ml | 3100 IU/ml | 3060 IU/ml |
| 16 | 3076 IU/ml | 3094 IU/ml | 3052 IU/ml | 16 | 3076 IU/ml | 3100 IU/ml | 3060 IU/ml |
| 17 | 3072 IU/ml | 3094 IU/ml | 3050 IU/ml | 17 | 3076 IU/ml | 3100 IU/ml | 3060 IU/ml |
| 18 | 3074 IU/ml | 3084 IU/ml | 3052 IU/ml | 18 | 3076 IU/ml | 3100 IU/ml | 3060 IU/ml |
| 19 | 3068 IU/ml | 3084 IU/ml | 3054 IU/ml | 19 | 3076 IU/ml | 3100 IU/ml | 3060 IU/ml |
| 20 | 3070 IU/ml | 3082 IU/ml | 3050 IU/ml | 20 | 3076 IU/ml | 3100 IU/ml | 3060 IU/ml |
| 21 | 3068 IU/ml | 3082 IU/ml | 3054 IU/ml | 31 | 3078 IU/ml | 3100 IU/ml | 3062 IU/ml |
| 22 | 3068 IU/ml | 3082 IU/ml | 3054 IU/ml | 32 | 3076 IU/ml | 3100 IU/ml | 3060 IU/ml |
| 23 | 3070 IU/ml | 3082 IU/ml | 3052 IU/ml | 33 | 3076 IU/ml | 3100 IU/ml | 3060 IU/ml |
| 24 | 3068 IU/ml | 3080 IU/ml | 3050 IU/ml | 34 | 3076 IU/ml | 3104 IU/ml | 3060 IU/ml |
| 25 | 3068 IU/ml | 3080 IU/ml | 3050 IU/ml | 35 | 3076 IU/ml | 3100 IU/ml | 3058 IU/ml |
| 26 | 3038 IU/ml | 3078 IU/ml | 3052 IU/ml | 36 | 3076 IU/ml | 3100 IU/ml | 3060 IU/ml |
| 27 | 3068 IU/ml | 3080 IU/ml | 3052 IU/ml | 37 | 3076 IU/ml | 3100 IU/ml | 3060 IU/ml |
| 28 | 3066 IU/ml | 3078 IU/ml | 3052 IU/ml | 38 | 3076 IU/ml | 3100 IU/ml | 3060 IU/ml |
| 29 | 3068 IU/ml | 3078 IU/ml | 3048 IU/ml | 39 | 3076 IU/ml | 3100 IU/ml | 3060 IU/ml |
| 30 | 3066 IU/ml | 3078 IU/ml | 3052 IU/ml | 40 | 3076 IU/ml | 3100 IU/ml | 3060 IU/ml |
| 31 | 3066 IU/ml | 3078 IU/ml | 3050 IU/ml | 41 | 3078 IU/ml | 3100 IU/ml | 3060 IU/ml |
| 32 | 3066 IU/ml | 3078 IU/ml | 3050 IU/ml | 42 | 3076 IU/ml | 3100 IU/ml | 3060 IU/ml |
| 33 | 3068 IU/ml | 3078 IU/ml | 3052 IU/ml | 43 | 3076 IU/ml | 3100 IU/ml | 3060 IU/ml |
| 34 | 3068 IU/ml | 3076 IU/ml | 3048 IU/ml | 44 | 3076 IU/ml | 3100 IU/ml | 3060 IU/ml |
| 35 | 3066 IU/ml | 3078 IU/ml | 3048 IU/ml | 45 | 3076 IU/ml | 3100 IU/ml | 3060 IU/ml |
| 36 | 3066 IU/ml | 3078 IU/ml | 3048 IU/ml | | | | |
| 37 | 3066 IU/ml | 3076 IU/ml | 3050 IU/ml | | | | |
| 38 | 3066 IU/ml | 3076 IU/ml | 3048 IU/ml | | | | |
| 39 | 3066 IU/ml | 3076 IU/ml | 3044 IU/ml | | | | |
| 40 | 3066 IU/ml | 3076 IU/ml | 3044 IU/ml | | | | |
| 41 | 3066 IU/ml | 3076 IU/ml | 3044 IU/ml | | | | |
| 42 | 3066 IU/ml | 3074 IU/ml | 3046 IU/ml | | | | |
| 43 | 3064 IU/ml | 3076 IU/ml | 3042 IU/ml | | | | |
| 44 | 3064 IU/ml | 3074 IU/ml | 3044 IU/ml | | | | |
| 45 | 3064 IU/ml | 3074 IU/ml | 3042 IU/ml | | | | |

The accelerated studies were conducted on multiple lots of the serine protease primary hemostatic agent at four temperatures. The lots were prepared in accordance with the description of Example 1, below. While for many hemostatic purposes a coagulant activity level of 1000 IU/ml is considered suitable, the tested agent is preferably formulated to a level of about 3000 IU/ml, and is considered to have reached the end of its shelf life when the activity level has degraded by more than about one-third of that amount.

Thus it can be seen from the accelerated testing that the serine protease agent has a shelf life of about one week at body temperature (37° C.), over one month at room temperature (25° C.), an undetermined time, but obviously more than 45 days when refrigerated to 2-4° C., and the coagulant activity level remains unchanged when the agent is frozen at 10° C. and thawed for daily testing before refreezing. As a result long term testing was undertaken for the refrigerated and freeze cycle storage of the hemostatic agent.

TABLE 5B
Long Term

| 2-4° C. | Lot 1 | Lot 2 | Lot 3 | −10° C. | Lot 1 | Lot 2 | Lot 3 |
|---|---|---|---|---|---|---|---|
| Day | Activity | Activity | Activity | Day | Activity | Activity | Activity |
| 60 | 3052 IU/ml | 3066 IU/ml | 3030 IU/ml | 60 | 3076 IU/ml | 3104 IU/ml | 3056 IU/ml |
| 90 | 3048 IU/ml | 3052 IU/ml | 3018 IU/ml | 90 | 3076 IU/ml | 3100 IU/ml | 3056 IU/ml |
| 120 | 3038 IU/ml | 3040 IU/ml | 3002 IU/ml | 120 | 3076 IU/ml | 3100 IU/ml | 3054 IU/ml |
| 150 | 3024 IU/ml | 3038 IU/ml | 2990 IU/ml | 150 | 3076 IU/ml | 3100 IU/ml | 3056 IU/ml |
| 180 | 3010 IU/ml | 3022 IU/ml | 2976 IU/ml | 180 | 3076 IU/ml | 3100 IU/ml | 3056 IU/ml |
| 210 | 2996 IU/ml | 3010 IU/ml | 2960 IU/ml | 210 | 3074 IU/ml | 3100 IU/ml | 3056 IU/ml |
| 240 | 2972 IU/ml | 2984 IU/ml | 2936 IU/ml | 240 | 3076 IU/ml | 3100 IU/ml | 3056 IU/ml |
| 270 | 2942 IU/ml | 2954 IU/ml | 2906 IU/ml | 270 | 3076 IU/ml | 3100 IU/ml | 3056 IU/ml |
| 300 | 2890 IU/ml | 2916 IU/ml | 2878 IU/ml | 300 | 3074 IU/ml | 3100 IU/ml | 3056 IU/ml |
| 330 | 2836 IU/ml | 2866 IU/ml | 2824 IU/ml | 330 | 3076 IU/ml | 3100 IU/ml | 3056 IU/ml |
| 360 | 2784 IU/ml | 2812 IU/ml | 2770 IU/ml | 360 | 3076 IU/ml | 3104 IU/ml | 3056 IU/ml |
| 390 | 2700 IU/ml | 2734 IU/ml | 2684 IU/ml | 390 | 3076 IU/ml | 3100 IU/ml | 3054 IU/ml |
| 420 | 2608 IU/ml | 2642 IU/ml | 2598 IU/ml | 420 | 3076 IU/ml | 3100 IU/ml | 3054 IU/ml |
| 450 | 2514 IU/ml | 2550 IU/ml | 2504 IU/ml | 450 | 3076 IU/ml | 3100 IU/ml | 3054 IU/ml |
| 480 | 2500 IU/ml | 2458 IU/ml | 2410 IU/ml | 480 | 3076 IU/ml | 3100 IU/ml | 3054 IU/ml |
| 510 | 2410 IU/ml | 2364 IU/ml | 2318 IU/ml | 510 | 3076 IU/ml | 3100 IU/ml | 3054 IU/ml |
| 540 | 2316 IU/ml | 2272 IU/ml | 2316 IU/ml | 540 | 3076 IU/ml | 3104 IU/ml | 3054 IU/ml |
| 570 | 2204 IU/ml | 2160 IU/ml | 2206 IU/ml | 570 | 3076 IU/ml | 3100 IU/ml | 3054 IU/ml |
| 600 | 2092 IU/ml | 2044 IU/ml | 2100 IU/ml | 600 | 3080 IU/ml | 3100 IU/ml | 3054 IU/ml |
| 630 | 1962 IU/ml | 1910 IU/ml | 1968 IU/ml | 630 | 3076 IU/ml | 3100 IU/ml | 3056 IU/ml |
| 660 | 2826 IU/ml | 1774 IU/ml | 1838 IU/ml | 660 | 3076 IU/ml | 3100 IU/ml | 3056 IU/ml |
| 690 | 1700 IU/ml | 1644 IU/ml | 1706 IU/ml | 690 | 3076 IU/ml | 3100 IU/ml | 3056 IU/ml |
| 720 | 1626 IU/ml | 1504 IU/ml | 1630 IU/ml | 720 | 3076 IU/ml | 3100 IU/ml | 3056 IU/ml |
| | | | | 750 | 3078 IU/ml | 3104 IU/ml | 3058 IU/ml |
| | | | | 780 | 3074 IU/ml | 3100 IU/ml | 3056 IU/ml |
| | | | | 810 | 3076 IU/ml | 3100 IU/ml | 3054 IU/ml |
| | | | | 840 | 3076 IU/ml | 3102 IU/ml | 3054 IU/ml |
| | | | | 870 | 3078 IU/ml | 3104 IU/ml | 3056 IU/ml |
| | | | | 900 | 3074 IU/ml | 3100 IU/ml | 3052 IU/ml |
| | | | | 930 | 3076 IU/ml | 3102 IU/ml | 3054 IU/ml |
| | | | | 960 | 3076 IU/ml | 3100 IU/ml | 3052 IU/ml |
| | | | | 990 | 3076 IU/ml | 3100 IU/ml | 3054 IU/ml |
| | | | | 1020 | 3072 IU/ml | 3100 IU/ml | 3056 IU/ml |
| | | | | 1050 | 3076 IU/ml | 3104 IU/ml | 3056 IU/ml |
| | | | | 1080 | 3074 IU/ml | 3104 IU/ml | 3052 IU/ml |
| | | | | 1110 | 3074 IU/ml | 3100 IU/ml | 3052 IU/ml |
| | | | | 1140 | 3074 IU/ml | 3100 IU/ml | 3054 IU/ml |
| | | | | 1170 | 3076 IU/ml | 3102 IU/ml | 3054 IU/ml |
| | | | | 1200 | 3078 IU/ml | 3100 IU/ml | 3052 IU/ml |
| | | | | 1230 | 3074 IU/ml | 3102 IU/ml | 3056 IU/ml |
| | | | | 1260 | 3076 IU/ml | 3100 IU/ml | 3056 IU/ml |
| | | | | 1290 | 3074 IU/ml | 3100 IU/ml | 3052 IU/ml |
| | | | | 1320 | 3078 IU/ml | 3104 IU/ml | 3054 IU/ml |
| | | | | 1350 | 3072 IU/ml | 3100 IU/ml | 3052 IU/ml |
| | | | | 1380 | 3074 IU/ml | 3104 IU/ml | 3052 IU/ml |
| | | | | 1410 | 3078 IU/ml | 3100 IU/ml | 3052 IU/ml |
| | | | | 1440 | 3076 IU/ml | 3102 IU/ml | 3052 IU/ml |

In the long term study, it can be seen that when refrigerated the serine protease hemostatic agent has a shelf life of over 18 months, and when subjected to freeze/thaw cycles over 48 months the agent lost little, if any, activity level.

### SUMMARY OF THE INVENTION

The present invention recognizes and addresses some of the foregoing disadvantages, and others, of prior art construction and methods.

Accordingly, it is an object of some embodiments of the present invention to provide a novel and improved method of controlling all forms of hemorrhages inside and outside of the clinical setting.

More particularly, it is an object of some embodiments of the present invention to be used as a primary hemostatic agent in place of surgical modalities when their application is limited or ineffective.

Another object of some embodiments of this invention is to achieve hemostasis without the loss of tissue, due to 3^{rd} degree burns from cauterization.

Other objects of some embodiments of this invention is to reduce blood loss significantly, and the need for blood transfusions, and to allow patients to maintain their anticoagulant therapy through surgical procedures.

Some embodiments of this invention may also reduce the overall surgical time, in turn reducing the quantity of anesthetic drugs administered during surgery.

Another object of some embodiments of this invention is to promote wound healing through the early presence of growth factors to the injured tissues.

The use of a stabilised serine protease hemostatic agent may reduce overall medical expenses through shorter surgical theater times, less anesthetic medication and post-operative pain medication, reduced need for blood transfusions and intensive care, and to generally shorten hospital stays.

The addition of a stabiliser reduces the oxidation, hydrolyzation, or other degradation of the coagulation activity of the serine protease hemostatic agent, and allows repetitive freeze-thaw cycles without substantial loss of coagulation activity.

### DESCRIPTION OF THE DRAWINGS

The invention, together with further objects and attendant advantages, will best be understood by reference to the following description, drawings, examples, and tables herein.

FIG. 1 is an agar structure illustrating linking of beta-galactopyranose with 3,6-anhydro-L-galactopyranose.

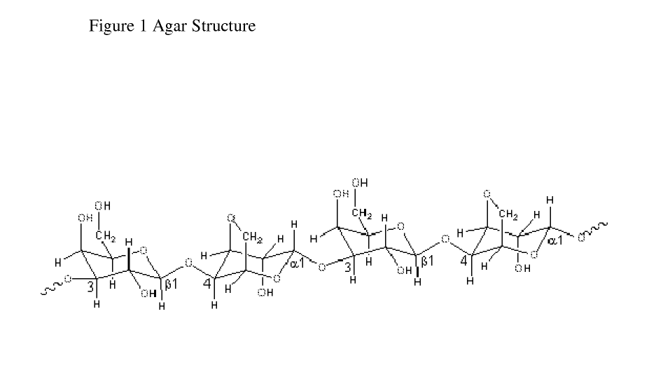

FIG. 2 illustrates effects of a primary serine protease hemostatic agent in the coagulation cascade system.

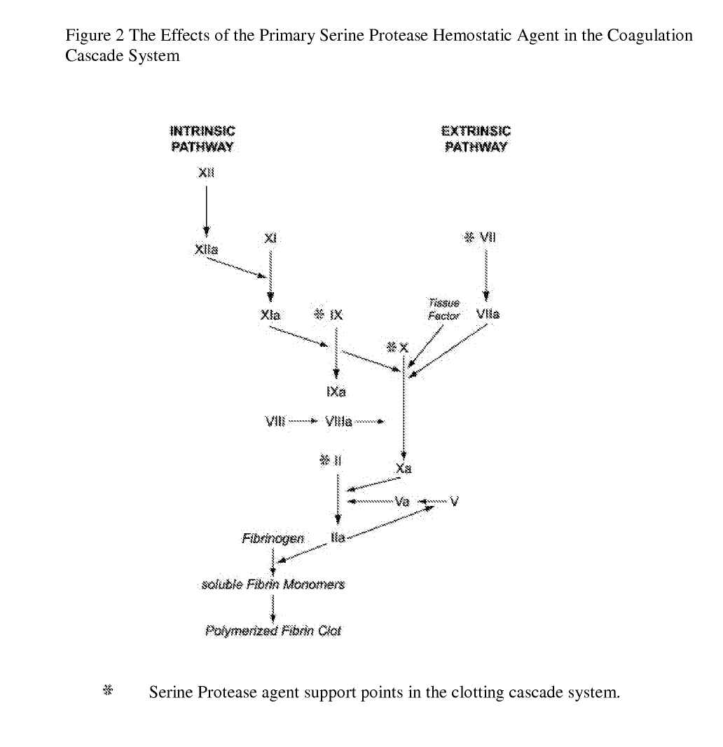

### DETAILED DESCRIPTION OF THE PREFERRED EMBODIMENTS

Reference will now be made in detail to a presently preferred embodiment of the invention. Disclosed is a method to stabilize labile serine proteases to be used as a primary hemostatic agent, and allowing the enzymes to be stored for lengthy periods in liquid form and to go through repetitive freeze-thaw cycles without significant loss of its activity.

The method begins with the isolation of Factors II, VII, IX, and X from 3.8%-4% sodium citrate plasma of either human, pig, horse, sheep, goat, or preferred bovine origin. There are a variety of industrial protein fractionation methods to isolate and purify each individual serine protease known to those skilled in the art of this technology. After the serine proteases have been isolated and purified, they require to be activated. There are a variety of methods to activate these specific enzymes by those skilled in the art of this technology. Some of these methods are described in Marjolis J., The Kaolin Clotting Time: a rapid one-stage method for diagnosis of coagulation defects. Journal of Clinical Pathology 1958; 11(5): 406-09.

Once the concentrated enzymes have been activated they are diluted with a diluent composed of a 0.001-1.0 M isotonic zwitter buffer from at least one of: sodium phosphate;

HEPBS (N-(2-Hydroxyethyl)piperazine-N′-(4-butanesulfonic acid));
HEPPSO (4-(2-Hydroxyethyl)piperazine-1-(2-hydroxypropanesulfonic acid)); Triethanolamine;
CAPSO (3-(Cyclohexylamino)-2-hydroxy-1-propanesulfonic acid);
CAPS (3-(Cyclohexylamino)-1-propanesulfonic acid);

### Imidaziole;

BES (2-[bis(2-hydroxyethyl)amino]ethanesulfonic acid);
BIS-TRIS (2-[Bis(2-hydroxyethyl)amino]-2-(hydroxymethyl)-1,3-propanediol);
2-amino-2-methyl-1,3-propanediol;

### Hydrazine;

### Pyrophosphate;

MOPSO (β-Hydroxy-4-morpholinepropanesulfonic acid, 3-Morpholino-2-hydroxypropanesulfonic acid);
MOPS (3-(N-Morpholino)propanesulfonic acid);
ACES (N-(2-Acetamido)-2-aminoethanesulfonic acid);
PIPES (piperazine-N,N′-bis(2-ethanesulfonic acid));
TAPS (N-[Tris(hydroxymethyl)methyl]-3-aminopropanesulfonic acid); or
TAPSO (2-hydroxy-3-[tris(hydroxymethyl) methylamino]-1-propanesulfonic acid).

The resulting liquid may have a pH of 1.0-12.0 as may be best suited for the particular buffer solution. To this is added 0.001-1000 mg/ml agar, and more preferentially 0.01-1.0 mg/ml agar, and most preferably 0.05-0.15 mg/ml agar. This mixture is used to dilute the serine protease enzymes to an activity level of 1-10,000 IU/ml, and more preferably about 2000-4000 IU/ml.

The diluent is most preferred to be composed of 0.05 M TAPSO, pH 7.41, with 0.1 mg/ml agar, and a sufficient volume of diluent added to serine protease concentrate to achieve a concentration level of Factor IIa to 0.1-1000 μg/ml, Factor VIIa 0.01-10 μg/ml, Factor IXa 0.1-100 μg/ml, and Factor Xa 0.1-1000 μg/ml, with a more preferred concentration level of Factor IIa 100-140 μg/ml, Factor VIIa 0.5-0.9 μg/ml, Factor IXa 5-9 μg/ml, and Factor Xa 10-14 μg/ml, that will produce a most preferred activity level of about 3000 IU/ml.

The targeted serine proteases are stabilized by the addition of 0.1-100 mg/ml of a stabilizer such as sodium iodine, sodium iodide, potassium iodine, povidone iodine, potassium iodide, di-iodohyroxyquinoline, piperazine citrate, iodochlorhydroxyquinoline, piperzaine hexahydrate, piperazine adipate, piperazine di-hydrochloride, piperazine phosphate, and binding substantially all of the stabilizer to the serine proteases and agar. The presently preferred stabilizer is about 1-10 mg/ml povidone iodine, and more preferably about 4-6 mg/ml povidone iodine. It is necessary that substantially all of the iodine be bound to the enzymes with no free iodine in solution. There are a variety of methods to bind povidone-iodine to the serine proteases and agar, to those skilled in the art of this technology. Marchaloins, J. An enzymic method for the trace iodination of immunoglobulins and other proteins. Biochem J. 113, 299-305(1969).

### Example 1

An exemplary primary hemostatic agent is mixed of:
(a) 0.1 mg/ml agar in a diluent containing 0.05 M TAPSO buffer, pH 7.42;
(b) Factor II, 120 μg/ml; Factor VII, 0.7 μg/ml; Factor IX, 7 μg/ml; Factor X12 μg/ml; and
(c) 5 mg/ml povidone iodine as a stabilizer.
(d) Such that the activity level of the resulting agent is about 3000 IU/ml MedLab Technol. 1973 October, 30(4): 387-90. Woods T F, Hill R H, Burnett D.
Comparison of a competitive protein binding method for serum thyroxine with a column technique for serum thyroxine iodine.

It will be apparent to those skilled in the art that medications and variations can be made in the present invention without departing from the scope or spirit thereof. For instance, although bovine plasma was used as the source of the serine proteases in Example 1, alternative plasmas can also be used. Thus, it is intended that the present invention cover such modifications and variations as come within the scope of the appended claims and their equivalents.

What is claimed is:

1. A hemostatic agent comprised of a liquid dilutent with a 0.001-1.0 M isotonic zwitter buffer comprising;
Factor IIa 0.1-1000 μg/ml;
Factor VIIa 0.01-10 μg/ml;
Factor IXa 0.1-100 μg/ml;
Factor Xa 0.1-1000 μg/ml;
0.001-1000 mg/ml agar; and
0.1-100 mg/ml stabilizer;
wherein the resulting mixture has an activity level between 1000 and 10,000 IU/ml and a shelf life greater than one year when refrigerated below 5° C.

2. The hemostatic agent of claim 1 wherein the activity level is not significantly degraded by 24 freeze/thaw cycles.

3. The composition of hemostatic agent of claim 1 wherein the activity level is not substantially degraded by 48 freeze/thaw cycles.

4. The hemostatic agent of claim 1 wherein the isotonic zwitter buffer is 0.01-0.1 M TAPSO buffer having pH between 6 and 9.

5. The hemostatic agent of claim 1 wherein the isotonic zwitter buffer is 0.03-0.08 M TAPSO buffer having pH between 7 and 8.

6. The hemostatic agent of claim 1 wherein the stabilizer is 1-10 mg/ml povidone iodine.

7. The hemostatic agent of claim 1 comprising between 0.01-1.0 mg/ml agar.

8. The hemostatic agent of claim 1 comprising between 0.05-0.15 mg/ml agar.

9. The hemostatic agent of claim 1 having an activity level between 2000 to 4000 IU/ml.

10. The hemostatic agent of claim 1 comprising:
100-140 μg/ml of Factor IIa;
0.5-0.9 μg/ml Factor VIIa;
5-9 μg/ml Factor IXa; and
10-14 μg/ml Factor Xa.

11. The hemostatic agent of claim 5 comprising 4-6 mg/ml povidone iodine.

12. A hemostatic agent comprised of a liquid dilutent with an isotonic zwitter buffer of 0.01-0.1 M TAPSO having pH between 6 and 9 comprising;
100-140 μg/ml of Factor IIa;
0.5-0.9 μg/ml Factor VIIa;
5-9 μg/ml Factor IXa; and
10-14 μg/ml Factor Xa.
0.05-0.15 mg/ml agar; and
4-6 mg/ml povidone iodine stabilizer;
wherein the resulting mixture has an activity level between 2000 and 4000 IU/ml and a shelf life greater than one year when refrigerated below 5° C.

13. The hemostatic agent of claim 12 having a shelf life greater than four years when frozen at or below −10° C.

14. The hemostatic agent of claim 13 wherein the activity level is not significantly degraded by 24 freeze/thaw cycles.

15. The composition of hemostatic agent of claim 13 wherein the activity level is not substantially degraded by 48 freeze/thaw cycles.

16. A method of effecting hemostasis at a bleeding wound site of a mammal in less than 3 seconds for venous hemorrhages comprising applying the composition of claim 12 to the wound site using a dropwise application of the hemostatic agent.

17. The method of claim 16 effecting hemostasis in less than 60 seconds for large anterial bleeds.
